(19) United States
(12) Patent Application Publication
(10) Pub. No.: US 2016/0077161 A1
(43) Pub. Date: Mar. 17, 2016

(54) METHOD FOR IMPROVED DIAGNOSTIC IN DETERMINING AND PREVENTING INVERTER FAULTS

(71) Applicant: EATON CORPORATION, Cleveland, OH (US)

(72) Inventors: Kenneth William Balogh, Minneapolis, MN (US); Greg John Venhaus, Arden, NC (US)

(73) Assignee: EATON CORPORATION, Cleveland, OH (US)

(21) Appl. No.: 14/484,337

(22) Filed: Sep. 12, 2014

(51) Int. Cl. G01R 31/40; G01N 33/00; G01K 13/00; G01R 33/02; G01H 17/00

(52) U.S. Cl. CPC G01R 31/40; G01R 33/02; G01H 17/00; G01K 13/00; G01N 33/0009

(57) ABSTRACT

The disclosed concept pertains to systems and methods for improving diagnostics in preventing inverter faults and determining the likelihood or occurrence of inverter faults. The disclosed concept includes employing one or more sensors capable of sensing and measuring one or more of magnetic field, temperature, vibration and humidity of the inverter. The measured values of these parameters are compared to previously obtained values or threshold values to determine whether action is required to preclude an inverter fault or cease operation in response to the occurrence of an inverter fault.

### BACKGROUND

1. Field

The disclosed concept pertains generally to inverters in power supply systems. In particular, the disclosed concept pertains to systems and methods for preventing and determining the occurrence of inverter faults.

2. Background Information

DC/AC power converters, e.g., inverters, are used in a variety of electrical systems, such as, without limitation, uninterruptible power supply systems and variable frequency drives. Their function is to convert a DC input voltage to an AC output voltage of desired amplitude and frequency. Inverter specifications include an input and an output voltage range, an output voltage frequency and a maximum output power. In conventional use, inverters operate within strict specifications, fail safely in the event of malfunction, record the inverter state and inform the supplied equipment and/or the operator about the cause of a failure.

It is known to employ special protection devices and control circuits to prevent faults from occurring and to determine whether a fault has occurred in an inverter. For example, it is common to monitor, e.g., sense, the inverter output current, load voltage and filter capacitor current, and compare these values to preset limits. If any of these quantities exceed the preset limits, an inhibit signal shuts off the DC power supply in order to prevent a fault from occurring. Further, there are known in the art various diagnostic systems and methods that may be employed to detect a fault that has occurred in an inverter.

The above-mentioned devices and methods do not fully detect all possible fault conditions. For example, current methods for preventing and determining if a fault has occurred in an inverter may include assessing phase voltage, current and gate driver feedback signals. These methods may not be successful in all instances of preventing a failure or of providing adequate information for determining if a failure has actually occurred. It is contemplated that additional sensors and data may result in improved methods both for preventing failure and determining if a failure has occurred.

Thus, it is an object of the disclosed concept to develop new monitoring and sensing systems and methods that provide additional sensors and data as compared to the traditional systems and methods in order to increase the likelihood that faults may be prevented from occurring in inventers and to increase the likelihood of determining when a fault has occurred in an inverter.

### SUMMARY

These needs and others are met by embodiments of the disclosed concept, which provide compositions and arc-resistant shields constructed of these compositions.

In an aspect, the disclosed concept provides a system for monitoring an inverter to prevent a fault from occurring or determining that a fault has occurred. The system includes one or more sensors selected from a magnetometer, vibration sensor, temperature sensor and humidity sensor to provide one or more measurements of magnetic field, vibration, temperature and humidity of the inverter, respectively; and a controller to receive the one or more measurements. A control value is determined for each of the one or more sensors such that when at least one of the one or more measurements exceeds the control value, corrective action is taken to prevent an occurrence of a fault or if the inverter inadvertently ceases operation, the one or more measurements is compared against the control value to determine if a fault occurred.

The control value can be selected from a pre-determined threshold value, a pre-set limit and an earlier obtained measured value.

The system may further include one or more additional sensors selected from phase voltage sensor, current sensor and gate driver sensor to generate additional inverter feedback. The additional inverter feedback can be received by the controller. The additional inventor feedback can be combined with the one or more measurements in preventing the occurrence of a fault or making the determination that a fault occurred.

In another aspect, the disclosed concept provides a method for monitoring an inverter for preventing a fault from occurring or for determining that a fault has occurred. The method includes implementing one or more sensors selected from magnetometer, vibration sensor, temperature sensor and humidity sensor; generating one or more measured values of the inverter selected from magnetic field, vibration, temperature and humidity; providing a controller to receive the one or more measured values; transferring the one or more measured values from the one or more sensors to the controller; establishing one or more control values to correspond to each of the one or more sensors; comparing the one or more measured values with the corresponding one or more control values; making a determination as to whether at least one of the one or more measured values reaches or exceeds the corresponding one or more control values; and initiating an action based on the determination.

### BRIEF DESCRIPTION OF DRAWINGS

A full understanding of the disclosed concept can be gained from the following description of the preferred embodiments when read in conjunction with the accompanying drawings in which:

FIG. 1 is a hardware block diagram showing the diagnostic processor and sensors, in accordance with certain embodiments of the disclosed concept;

FIG. 2 is a schematic of data collection tasks, in accordance with certain embodiments of the disclosed concept; and

FIGS. 3 and 4 are schematics showing fault diagnostics using sensor inputs, in accordance with certain embodiments of the disclosed concept.

### DETAILED DESCRIPTION OF PREFERRED EMBODIMENTS

The disclosed concept includes systems and methods for monitoring, e.g., sensing, reading and measuring, conditions of DC/AC power converters, e.g., inverters, which are used in a variety of applications, such as, without limitation, uninterruptible power supply systems and variable frequency drives, to prevent a fault from occurring and/or determining that a fault has occurred. The systems include one or a plurality of sensors that sense, read and measure inverter data, e.g., signals, as input to provide information relating to the conditions and/or change of conditions within the inverter. One or more of the following sensors are provided in accordance with the disclosed concept: a magnetometer, a vibration sensor (e.g., an accelerometer), a temperature sensor and a humidity sensor. Each of these sensors monitors and provides measurements of magnetic field, vibration, temperature and humidity, respectively. A preset limit or threshold value is determined for each of the sensors. The sensor data is assessed and a diagnostic is performed. Based on the assessment and diagnostic, the likelihood of a future fault or failure occurring may be determined, and/or a determination as to whether a fault or failure has actually occurred can be made. For example, when one or more of the sensors indicates that a measured value exceeds the preset limit or threshold value, an action is performed. In certain embodiments, an alarm may be generated to alert of a potential for the occurrence of a fault or in certain other embodiments, operation of the inverter may be stopped in order to prevent an occurrence of a fault. In embodiments wherein the inverter inadvertently ceases operation, one or more of the measured values of magnetic field, vibration, temperature and humidity are compared against the present limits or threshold values to determine if a fault occurred.

It is contemplated that the sensors and the information obtained therefrom may be employed alone or in combination with one or more of the traditional and conventional inverter sensors which are employed to read and measure phase voltage, current and gate driver feedback signals.

In accordance with certain embodiments of the disclosed concept, a humidity sensor subsystem provides the ability to measure conditions that may lead to condensation forming inside a drive. Under certain conditions, condensation can lead to arcing. Medium voltage components, in particular, are at a higher risk for arcing, which can lead to component failure and permanent damage. An internal system that is capable of detecting condensation, e.g., a threshold level of condensation, will allow the drive to shut down before damage occurs.

Accelerometers, which are typically located on a main controller, are potentially useful throughout the lifecycle of a device employing an inverter, such as, a variable frequency drive. Conventional accelerometers are capable of measuring amplitude and direction of vibration. During transportation of the drive, high vibration can lead to damage, such as in the form of loosened mechanical fasteners, loosened screw terminal connections, and damage to components or solder connections on circuit boards. In accordance with the disclosed concept, this system is embedded in the device employing an inverter, such as, a variable frequency drive, and allows for notification to the installers that vibration limits have been exceeded before the unit is powered for the first time.

In addition to the transportation phase, real time shock monitoring will allow devices to monitor seismic activity, enabling shut down of itself in the event of an earthquake. Thus, avoiding possible damage to the device, such as, a variable frequency drive, or the mechanical components being driven by the motor that the drive is controlling.

In accordance with certain other embodiments of the disclosed concept, digital signal processing of accelerometer data allows a device employing an inverter, such as, a variable frequency drive, to look for specific frequency components and find the vibration signatures of bearings or lamination stacks, which can enable the detection of blower, fan, or power transformer failures.

Furthermore, it is known in the art that atmospheric pressure is useful to determine changes in the weather. For example, a drop in pressure may correspond to conditions of elevated humidity and/or rain. In certain embodiments of the disclosed concept, a pressure measurement subsystem allows a device employing an inverter to detect a drop in pressure and anticipate a change in weather. This allows the device to shut down in advance of a high humidity situation to avoid damage resulting from condensation.

In certain embodiments of the disclosed concept the magnetometer, vibration, temperature and humidity sensors are all employed to sense and read inverter feedback, e.g., signals, and the information obtained from each of the sensors is considered alone or in combination to assess inverter fault conditions. However, it is also contemplated that in certain embodiments, less than all four of these sensors are employed. Further, the quantity of each type of sensor or the total number of sensors may vary and depend upon the size of the inverter to be monitored.

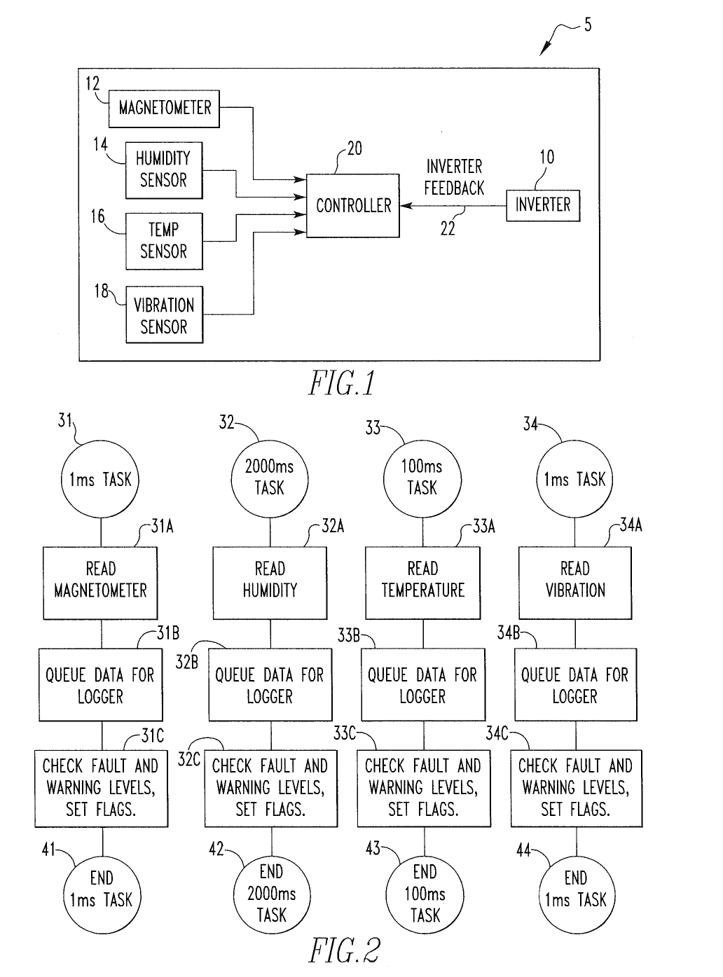

FIG. 1 is a hardware block diagram showing a power supply system 5 in accordance with certain embodiments of the disclosed concept. The system 5 includes an inverter 10. As previously described, it is contemplated that in certain other embodiments, the system 5 may be a variable frequency drive and the inverter 10 may be another DC/AC power converter. As shown in FIG. 1, an inverter monitoring system according to the disclosed concept includes a magnetometer 12, a humidity sensor 14, a temperature sensor 16 and a vibration sensor 18. These sensors can be positioned in various locations within the system 5, such as but not limited to, in close proximity to the inverter 10. Further, it is contemplated that in certain embodiments not all four of the sensors 12, 14, 16 and 18 are present. For example, one or two or three of these sensors may be employed. The sensors 12,14,16,18 may be selected from a wide variety of conventional sensors that are known in the art to provide a corresponding measured value or reading. In the exemplary embodiment, the magnetometer 12 measures and provides a magnetic field reading, the humidity sensor 14 measures and provides a humidity reading, the temperature sensor 16 measures and provides a temperature reading, and the vibration sensor 18 measures and provides a vibration reading from the inverter 10. Each of the sensors 12, 14, 16 and 18 are connected to a controller 20. The readings from each of these sensors are provided to, and received by, the controller 20. The controller 20 may be selected from a wide variety of conventional controllers known in the art, such as, but not limited to, microprocessors, Digital Signal Processing (DSP) controllers, microcontrollers and Field-Programmable Gate Array (FPGA) controllers. The controller 20 includes a threshold value or pre-set limit (stored by controller 20 or a memory associated with controller 20) which corresponds to each of the sensors 12,14,16,18. In the controller 20, the measured value or reading for each of the sensors 12,14,16,18 is compared to the corresponding threshold value or pre-set limit. Based on the comparison, it is determined whether one or more of the measured values or readings for the sensors 12,14,16,18 exceeds the corresponding threshold value or pre-set limit. When the measured values or readings derived from the sensors 12,14,16,18 reaches or exceeds the preset limit or threshold value for at least one of the sensors, an action is performed. The action can include initiating an alarm or implementing shutdown of the inverter 10.

In certain embodiments, sensors 12,14,16,18 may be “smart” sensors which term is used herein to refer to sensors have a threshold value or pre-set limit programmed or set therein such that the sensors 12,14,16,18 provide notification to the controller 20 when the threshold value or pre-set limit is reached or exceeded.

In addition, inverter feedback 22 is provided as input to the controller 20. The inverter feedback 22 may be considered in combination with the readings of the sensors 12,14,16,18 to assess the likelihood of a fault occurring or whether a fault has occurred. Further, each of the measured values may be compared to previously obtained measured values (stored by controller 20 or a memory associated with controller 20) to determine a change in at least one of magnetic field, humidity, temperature and vibration, and based on the change(s), an action can be performed.

Thus, based on the measured values or readings of the monitoring sensors 12,14,16,18, the disclosed concept is capable of sensing conditions relating to a potential, future fault occurrence and taking action for avoidance. Further, the disclosed concept is capable of determining if a fault occurrence has taken place in the event that inverters inadvertently ceases operation.

The hardware described and included in certain embodiments of the disclosed concept requires a minimum level of support firmware. The sensors 12,14,16,18 which provide data that is used for fault detection, prediction and warning can be sampled at various intervals. The type of information measured by the sensors can dictate how often the data is sampled. For example, ambient temperature and humidity are parameters that generally do not change rapidly and therefore, do not require a high sampling rate. In contrast, vibration and magnetic field are parameters that may change relatively rapidly and therefore, are sampled at a more frequent rate (as compared to the temperature and humidity).

FIG. 2 is a schematic showing individual data collection tasks in accordance with certain embodiments of the disclosed concept. Tasks 31 through 34 represent individual data collection tasks which correspond to the magnetometer 12, the humidity sensor 14, the temperature sensor 16 and the vibration sensor 18, respectively. Each of the tasks 31 through 34 include steps A, B and C. In steps 31A, 32A, 33A and 34A, the corresponding sensor, i.e., 12,14,16,18, respectively, is read. Steps 31B, 32B, 33B and 34B, recite queuing data for logger and steps 31C, 32C, 33C and 34C each recite checking fault and warning levels and setting flags. In steps 41, 42, 43 and 44, each of the data collection tasks are ended. It is contemplated that the task times can be either faster or slower than the times identified in FIG. 2.

The data collection tasks can remain dormant until triggered or initiated by a time-based interrupt. For example, a timer interrupt can be used to optimize the amount of the time the system processor spends collecting data, and to collect the data in a regular, deterministic manner. It also allows the system process to use the remaining time for other foreground functions.

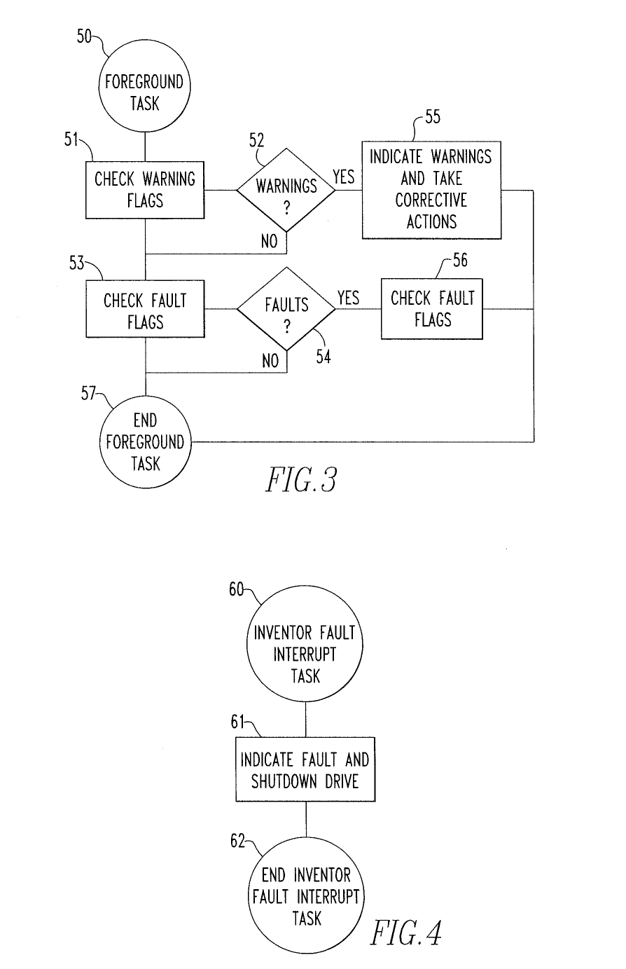

FIG. 3 is a schematic showing a routine that may be executed, e.g., in the remaining time that is allotted to the system processor. The routine includes a polled task 50 running in the foreground. In step 51, warning flags (set by tasks 31-34 shown in FIG. 2) are checked and in step 53, fault flags (set by tasks 31-34 shown in FIG. 2) are checked. In steps 52 and 54, respectively, it is determined if any warning flags or fault flags 54 are detected. If yes, steps 55 and 56, respectively, indicate a warning or fault threshold has been reached or exceeded and corrective action is taken. In certain embodiments, appropriate corrective action may include shutting down the drive. In step 57, the foreground task is ended.

FIG. 4 is a schematic showing a higher priority task that is driven by a direct hardware interrupt, so as to take as soon as possible if a high priority inverter fault occurs. Task 60 shows an inverter fault interrupt. In step 61 a fault is indicated and the drive is shutdown, and step 62 ends the inverter fault interrupt task.

While example systems, methods, and the like have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the disclosed concept is not limited to the specific details, the representative apparatus, and illustrative examples shown and described. Thus, this application is intended to embrace alterations, modifications, and variations that fall within the scope of the appended claims.

What is claimed is:

1. A system for monitoring an inverter to prevent a fault from occurring or determining that a fault has occurred, comprising:
one or more sensors selected from a magnetometer, vibration sensor, temperature sensor and humidity sensor to provide one or more measurements of magnetic field, vibration, temperature and humidity of the inverter, respectively; and
a controller to receive the one or more measurements;
wherein a control value is determined for each of the one or more sensors such that when at least one of the one or more measurements exceeds the control value, corrective action is taken to prevent an occurrence of a fault or
wherein the inverter inadvertently ceases operation, the one or more measurements is compared against the control value to determine if a fault occurred.

2. The system of claim 1, wherein the control value is selected from a pre-determined threshold value, a pre-set limit and an earlier obtained measured value.

3. The system of claim 1, wherein four sensors are employed consisting of a magnetometer, vibration sensor, temperature sensor and humidity sensor.

4. The system of claim 1, wherein the controller is selected from the group consisting of DSP, Micro and FPGA.

5. The system of claim 1, further comprising one or more additional sensors selected from phase voltage sensor, current sensor and gate driver sensor to generate additional inverter feedback.

6. The system of claim 5, wherein the additional inverter feedback is received by the controller.

7. The system of claim 6, wherein the additional inventor feedback is combined with the one or more measurements in preventing the occurrence of a fault or making the determination that a fault occurred.

8. The system of claim 1, wherein it is determined that a fault has occurred based on the vibration measured value exceeding the vibration threshold value, a change in the magnetic field and the temperature measured value exceeding the temperature threshold value.

9. A method for monitoring an inverter for preventing a fault from occurring or for determining that a fault has occurred, comprising:
implementing one or more sensors selected from magnetometer, vibration sensor, temperature sensor and humidity sensor;
generating one or more measured values of the inverter selected from magnetic field, vibration, temperature and humidity;
providing a controller to receive the one or more measured values;
transferring the one or more measured values from the one or more sensors to the controller;
establishing one or more control values to correspond to each of the one or more sensors;
comparing the one or more measured values with the corresponding one or more control values;
making a determination as to whether at least one of the one or more measured values reaches or exceeds the corresponding one or more control values; and
initiating an action based on the determination.

10. The method of claim 9, wherein the control value is selected from a pre-determined threshold value, a pre-set limit and an earlier obtained measured value.

11. The method of claim 10, wherein the measured value of one or more of magnetic field, vibration, temperature and humidity exceeds the threshold value of the one or more sensors and operation of the inverter is stopped to prevent an occurrence of a fault.

12. The method of claim 10, wherein the inverter inadvertently ceases operation and the measured value of one or more of magnetic field, vibration, temperature and humidity is compared against the threshold value of the one or more sensors to determine if a fault occurred.
